(19) United States
(12) Patent Application Publication
(10) Pub. No.: US 2024/0186019 A1
(43) Pub. Date: Jun. 6, 2024

(54) DUAL ANTIPLATELET THERAPY AND TIME BASED RISK PREDICTION

(71) Applicant: Abbott Laboratories, Abbott Park, IL (US)

(72) Inventors: Divine E. Ediebah, San Francisco, CA (US); Jana R. Buccola, Rocklin, CA (US); Ciaran Byrne, San Francisco, CA (US)

(21) Appl. No.: 18/514,454

(22) Filed: Nov. 20, 2023

Related U.S. Application Data
(60) Provisional application No. 63/385,696, filed on Dec. 1, 2022.

(51) Int. Cl. G16H 50/30; G16H 50/20; G16H 50/70

(52) U.S. Cl. CPC G16H 50/30; G16H 50/20; G16H 50/70

(57) ABSTRACT

Systems, apparatuses and methods may provide technology that automatically converts, by a machine learning model, a Shapley plot into a hazard ratio plot. The technology may also identify a set of preoperative baseline characteristics associated with a procedure on a pooled patient population, determine, by a machine learning model, a set of health failure probabilities for a target patient based on the set of preoperative baseline characteristic and a set of preoperative target characteristics, wherein the set of preoperative target characteristics correspond to the target client, and pair, by the machine learning model, each probability in the set of health failure probabilities with a postoperative dual antiplatelet therapy (DAPT) duration for the target patient.

### CROSS-REFERENCE TO RELATED APPLICATIONS

The present application claims the benefit of priority to U.S. Provisional Patent Application No. 63/385,696 filed on Dec. 1, 2022, which is incorporated herein in its entirety by reference.

### TECHNICAL FIELD

Embodiments generally relate to risk predictions in clinical medicine. More particularly, embodiments relate to dual antiplatelet therapy (DAPT) and time based risk prediction in clinical medicine.

### BACKGROUND

Percutaneous coronary intervention (PCI, e.g., coronary angioplasty with stent), is a nonsurgical procedure that improves blood flow to the heart. Target lesion failure (TLF) is a health failure (e.g., heart attack, cardiac death) related to the vessel targeted in a PCI. Recent developments have shown the potential ability to use machine learning to identify the most important physiological variables that contribute to a future patient risk for events such as TLF. There remains room for improvement, however, with respect to the reliability of risk predictions.

### SUMMARY

In accordance with one or more embodiments, a computing system comprises a processor and a memory coupled to the processor, the memory including a set of instructions, which when executed by the processor, cause the computing system to identify a set of preoperative baseline characteristics associated with a procedure on a pooled patient population, determine, by a machine learning model, a set of health failure probabilities for a target patient based on the set of preoperative baseline characteristics and a set of preoperative target characteristics, wherein the set of preoperative target characteristics correspond to the target patient, and pair, by the machine learning model, each probability in the set of health failure probabilities with a postoperative dual antiplatelet therapy (DAPT) duration for the target patient.

In accordance with one or more embodiments, at least one computer readable storage medium comprising a set of instructions, which when executed by a computing system, cause the computing system to identify a set of preoperative baseline characteristics associated with a procedure on a pooled patient population, determine, by a machine learning model, a set of health failure probabilities for a target patient based on the set of preoperative baseline characteristics and a set of preoperative target characteristics, wherein the set of preoperative target characteristics correspond to the target patient, and pair, by the machine learning model, each probability in the set of health failure probabilities with a postoperative dual antiplatelet therapy (DAPT) duration for the target patient.

In accordance with one or more embodiments, a method comprises identifying a set of preoperative baseline characteristics associated with a procedure on a pooled patient population, determining, by a machine learning model, a set of health failure probabilities for a target patient based on the set of preoperative baseline characteristics and a set of preoperative target characteristics, wherein the set of preoperative target characteristics correspond to the target patient, and pairing, by the machine learning model, each probability in the set of health failure probabilities with a postoperative dual antiplatelet therapy (DAPT) duration for the target patient.

In accordance with one or more embodiments, a computing system comprises a processor and a memory coupled to the processor, the memory including a set of instructions, which when executed by the processor, cause the computing system to generate, by a machine learning model, a Shapley plot based on average marginal contributions of a group of patients to a plurality of variables, conduct a conversion of a portion of the Shapley plot into a hazard ratio value, wherein the hazard ratio value is a single value corresponding to a first variable in the plurality of variables, and generate a hazard ratio plot based at least in part on the hazard ratio value.

In accordance with one or more embodiments, at least one computer readable storage medium comprising a set of instructions, which when executed by a computing system, cause the computing system to generate, by a machine learning model, a Shapley plot based on average marginal contributions of a group of patients to a plurality of variables, conduct a conversion of a portion of the Shapley plot into a hazard ratio value, wherein the hazard ratio value is a single value corresponding to a first variable in the plurality of variables, and generate a hazard ratio plot based at least in part on the hazard ratio value.

In accordance with one or more embodiments, a method comprises generating, by a machine learning model, a Shapley plot based on average marginal contributions of a group of patients to a plurality of variables, conducting a conversion of a portion of the Shapley plot into a hazard ratio value, wherein the hazard ratio value is a single value corresponding to a first variable in the plurality of variables, and generating a hazard ratio plot based at least in part on the hazard ratio value.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The various advantages of the embodiments will become apparent to one skilled in the art by reading the following specification and appended claims, and by referencing the following drawings, in which:

FIG. 1A is a chart of an example of average marginal contributions of a group of patients to a plurality of variables associated with ischemic events according to an embodiment;

FIG. 1B is a chart of an example of average marginal contributions of a group of patients to a plurality of variables associated with bleeding events according to an embodiment;

FIG. 2 is a chart of an example of a plurality of Shapley plots according to an embodiment;

FIG. 3 is a chart of an example of a plurality of hazard ratio plots according to an embodiment;

FIG. 4A is a flowchart of an example of a method of plotting hazard ratios according to an embodiment;

FIG. 4B is a flowchart of an example of a method of converting a Shapley plot into a hazard ratio plot according to an embodiment;

FIG. 5A is an illustration of an example of pairings between health failure probabilities and DAPT durations for a patient having no health failure events according to an embodiment;

FIGS. 5B and 5C are illustrations of an example of patient specific variable components that contribute to predicted risk for a patient having no health failure events according to an embodiment;

FIG. 6A is an illustration of an example of pairings between health failure probabilities and DAPT durations for a patient having health failure events according to an embodiment;

FIG. 6B is a set of illustrations of an example of patient specific variable components that contribute to predicted risk for a patient having an ischemic health failure according to an embodiment;

FIG. 6C is a set of illustrations of an example of patient specific variable components that contribute to predicted risk for a patient having a bleeding health failure according to an embodiment;

FIG. 7 is a flowchart of an example of a method of recommending DAPT durations according to an embodiment; and

FIG. 8 is a block diagram of an example of a computing system according to an embodiment;

FIG. 9 is a comparative plot of an example of conventional prediction results and prediction results according to an embodiment;

FIG. 10A is a comparative plot of an example of conventional time-dependent area under curve (tAUC) results for bleeding events and tAUC results for bleeding events according to an embodiment;

FIG. 10B is a comparative plot of an example of conventional tAUC results for ischemic events and tAUC results for ischemic events according to an embodiment;

FIG. 10C is a comparative plot of an example of conventional tAUC results for ischemic events and tAUC results for ischemic events according to an embodiment in which the top four preoperative baseline characteristics were used to determine the health failure probabilities;

FIG. 10D is a comparative plot of an example of conventional tAUC results for ischemic events and tAUC results for ischemic events according to an embodiment in which the top three preoperative baseline characteristics were used to determine the health failure probabilities; and

FIG. 10E is a plot of an example of individual risk scores for a plurality of variables according to an embodiment.

### DESCRIPTION OF EMBODIMENTS

Machine learning models have gained increasing attention in clinical medicine because of their advantages to incorporate multiple independent variables to yield more accurate predictions for future event rates and patient survival. As already noted, previous approaches have shown the potential for machine learning technology to identify the most important physiological variables that contribute to a future patient risk for particular events such as, for example, a Target Lesion Failure (TLF). For that analysis, patent-level data was pooled for a variety of variables such as, for example, age, body mass index (BMI), reference vessel diameter (RVD), stent length used, and procedural time involved. In all, over eighty patient level variables were reduced down to the ten most significant contributors for future risk prediction by employing machine learning technology (e.g., Random Forest, Extra Tree classifier, neural networks) and when needed, synthetic minority oversampling techniques (SMOTE, e.g., to generate sufficient data for underrepresented events).

The reduced set of risk factor variables enable physicians to gather and track the needed information more easily. These reduced set of ten input variables are then fed into the risk prediction models that predict with more than seventy percent accuracy, specificity and sensitivity compared to using the eighty variable data set. The predictive ability from the machine learning results were also superior to traditional linear regression models. These predictive results were independent, however, of the time going forward so that the risk for TLF would be a certain set figure that did not increase or decrease. This inelastic prediction was based on the binary nature of the input data—whether a TLF event did or did not happen at the conclusion of one year irrespective of when the event actually occurred during that year interval.

The technology described herein extends the use of machine learning to plot hazard ratios (e.g., more effectively visualizing significant contributors for future risk prediction) and recommend dual antiplatelet therapy (DAPT) durations for individual/target patients (e.g., achieving time based risk prediction). DAPT, which is a treatment to prevent harmful blood clots from forming, typically involves taking two types of antiplatelet medicines—aspirin and a P2Y12 inhibitor. Clinical data has indicated that the majority of health failure events (e.g., major adverse cardiovascular events/MACE) occur while a patient is on DAPT. These advances therefore significantly improve performance and lead to better patient outcomes.

More particularly, embodiments are also based upon inputting patient variables and procedure baseline characteristics, but the output is risk probabilities for both an ischemic event occurring and the risk of a bleeding event. A difference compared to previous approaches is that the risk probabilities are now time based as a co-variate when modeled using machine survival learning. Below are the various clinical studies used to obtain anonymized, pooled patient data that contained approximately 19,000 patients.

TABLE I

| | | 1 m | 3 m | 6 m | 12 m |
|---|---|---|---|---|---|
| Study | N | DAPT | DAPT | DAPT | DAPT |
| XV USA | 8040 | — | — | — | X |
| X28 | 1605 | X | — | — | — |
| X90 | 2047 | — | X | — | — |
| COMPARE | 885 | — | — | — | X |
| Acute | | | | | |
| COMPARE | 822 | — | — | X | X |
| Absorb | | | | | |
| ABSORB III | 686 | — | — | — | X |
| ABSORB IV | 1308 | — | — | — | X |
| EXAMINATION | 750 | — | — | — | X |
| SIERRA-75 | 686 | X | X | X | X |

These studies had the prerequisite data of patients being prescribed a DAPT duration of 1, 3, 6 or 12 months (columns 5-8) and concurrent monitoring of resulting ischemia and/or bleeding incidences (columns 3 and 4) during the 12-month follow-up time and noting when those events occurred. Bleeding event risk was defined by the Bleeding Academic Research Consortium (BARC) types 3-5. Ischemic event risk was the composite of any of the following events: cardiovascular death, myocardial infarction (any type), stroke and stent thrombosis.

For model development, 75% of the patient data may be used for machine learning training, with the remaining 25% of the patient data being retained for validation. In one example, an ischemic event rate of 6.4% for approximately 11,000 patients sampled was observed. This minority class of 6.4% is imbalanced for developing predictive models as there are too few examples of this dataset during the machine learning process. Therefore, oversampling the minority class can be achieved by developing synthetic new minority class data (e.g., synthetic minority oversampling technique /SMOTE). After employing SMOTE, a 43% ischemic event rate was obtained (e.g., sufficient for machine learning analysis).

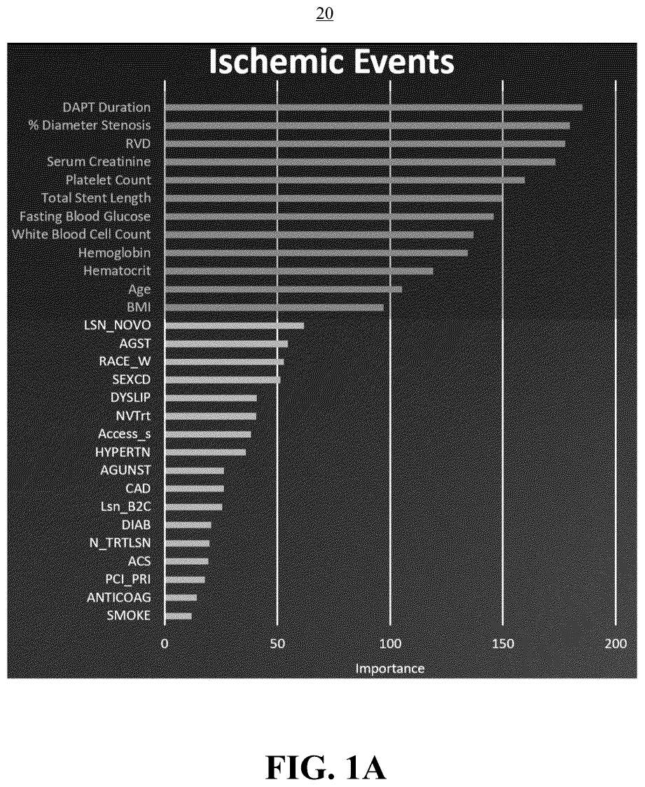

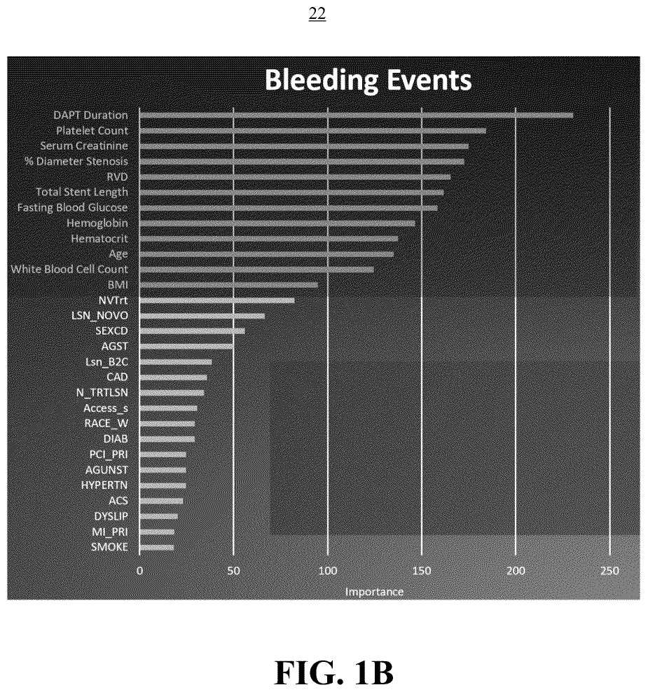

Turning now to FIGS. 1A and 1B, a chart 20 is shown of average marginal contributions (e.g., relative importance, entropy) of a group of patients (e.g., pooled patient population) to a plurality of variables associated with ischemic events. In the illustrated example, the DAPT duration, percent diameter stenosis (e.g., worst case percentage diameter stenosis of all lesions before being treated within PCI) and RVD (reference vessel diameter, e.g., worst case reference vessel diameter defined by minimum RVD of treated lesions in PCI) are the most important variables when predicting ischemic events. Similarly, a chart 22 demonstrates that DAPT duration, platelet count (e.g., measured at baseline), and serum creatinine (e.g., measured at baseline) are the most important variables when predicting bleeding events. In an embodiment, the charts 20, 22 are generated by iteratively/repeatedly (e.g., 100 iterations) considering original variables and a randomized version of the variables to determine variable importance for the occurrence of the event.

### Variable Selection—Boruta Procedure

More particularly, Boruta automation may operate as a “wrapper” around Random Forest machine learning technology. In Boruta, variables do not compete among themselves. Rather, variables compete with a randomized version of themselves or shuffled copies, which are called shadow variables. The Boruta procedure then trains a classifier or survival model (e.g., Random Forest) on the data set and applies a variable importance measure such as, for example, Mean Decrease Accuracy (e.g., a test statistic that is the mean decrease of accuracy of trees divided by the standard deviation) to evaluate the importance of each variable, where a higher value corresponds to greater importance. At every iteration, the Boruta procedure checks whether a real variable has a higher importance measure than the best of the corresponding shadow variables (e.g., whether the variable has a higher Z-score than the maximum Z-score of the corresponding shadow variables) and constantly removes variables that are deemed highly unimportant. Finally, the Boruta procedure stops either when all variables are confirmed or rejected or a specified limit of random forest runs is reached.

The importance of the original variable is then compared with a threshold defined as the highest variable importance recorded among the shadow variables. When a variable is greater than the threshold, the variable is considered a “hit”. Thus, a variable is flagged as important only if the variable is scores better than the randomized version or the respective shadow variable. Table I below shows an example of randomized/shuffled data and Table II below shows the resulting hit determinations (e.g., with age and height performing better than their respective shadow variable, but weight not performing better than its respective shadow variable).

TABLE I

| | | | | shad- | shad- | shad- |
|---|---|---|---|---|---|---|
| | age | height | weight | ow_age | ow_height | ow_weight |
| 0 | 25 | 182 | 75 | 51 | 176 | 75 |
| 1 | 32 | 176 | 71 | 32 | 182 | 71 |
| 2 | 47 | 174 | 78 | 47 | 168 | 78 |
| 3 | 51 | 168 | 72 | 25 | 181 | 72 |
| 4 | 62 | 181 | 86 | 62 | 174 | 86 |

TABLE II

| | | | | shad- | shad- | shad- |
|---|---|---|---|---|---|---|
| | age | height | weight | ow_age | ow_height | ow_weight |
| feature | 39 | 19 | 8 | 11 | 14 | 9 |
| impor- | | | | | | |
| tance % | | | | | | |
| hits | 1 | 1 | 0 | — | — | — |

The Boruta procedure may be implemented with a binomial distribution. For example, iterations may be based on a reliability and decision criterion (e.g., twenty trials versus one trial, with 100 trials being more reliable than twenty trials). Table III below shows an example number of hits for each variable in twenty trials.

TABLE III

| | age | height | weight |
|---|---|---|---|
| hits (in 20 trials) | 20 | 4 | 0 |

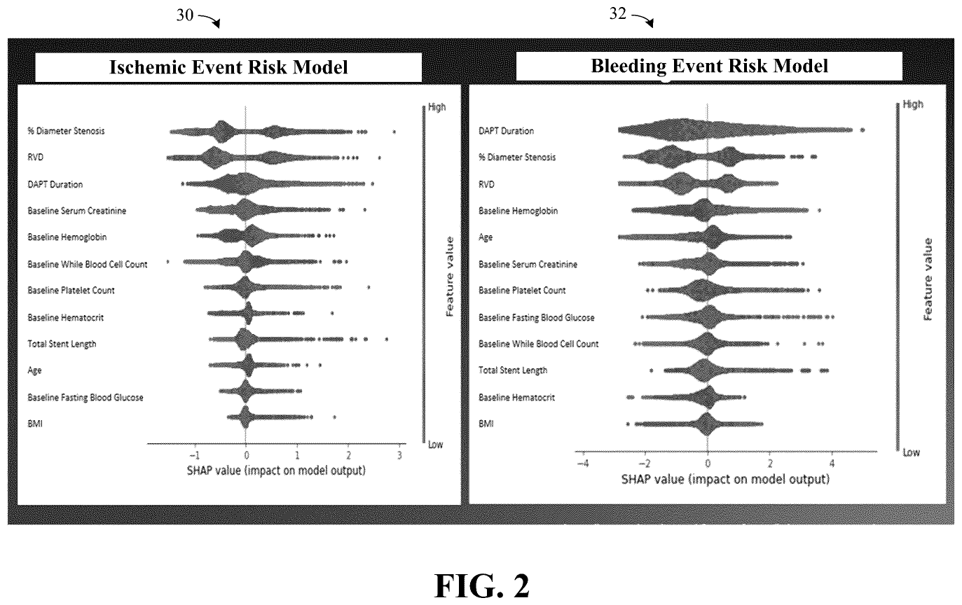

FIG. 2 demonstrates that a machine learning model (e.g., Random Survival Forests, Gradient Boosting) may use the average marginal contributions from the charts 20, 22 (FIGS. 1A and 1B) to automatically generate Shapley plots 30, 32 based on the SHAP (SHapley Additive explanations) method, which is a method used to explain individual predictions. More particularly, for each patient covariate data point in both the ischemic and bleeding event risk models, the SHAP method converts the average marginal contribution as a Shapley value point. The Shapley plots 30, 32 determine the variables that have the greatest influence on the predictive risk. The variables that contribute the most to the predictive risk are at the top of the plots 30, 32 while the lowest are at the bottom of the plots 30, 32. For ischemic event risk, the influential contributor was the percentage diameter of stenosis followed by reference vessel diameter (RVD) and then DAPT duration for the top three variables. For bleeding event risk, DAPT duration was the major contributor followed by percentage Diameter of Stenosis followed by reference vessel diameter (RVD).

All dot values on the left represent observations that shift the predictive value of that point in the negative direction while the points on the right shift the prediction in the positive direction (e.g., each dot represents a patient). Blue dots are associated with lower risk values for that particular classification, whereas red dots are higher risk. For example, in the ischemic event Shapley plot 30, on average an increased DAPT duration (blue dots) contributed to a lowering of future Ischemic events (right dot position shifting and lowering the predictive event value). Similarly, on average increased DAPT duration (blue dots) contributed to a lowering of future bleeding risk in the bleeding event Shapley plot 32.

### Conversion of Shapley Value Plots to Hazard Ratios

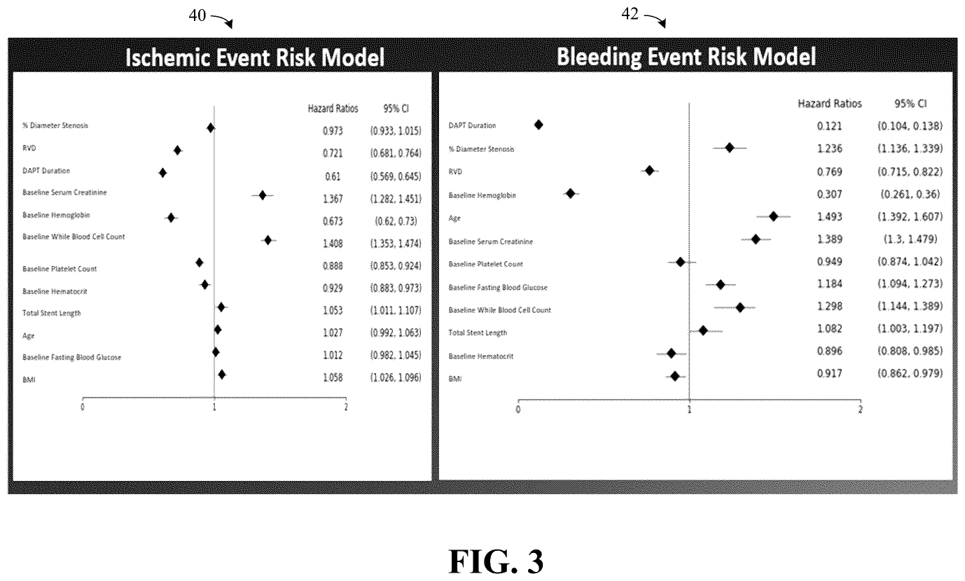

Turning now to FIG. 3, an ischemic event hazard plot 40 and a bleeding event hazard plot 42 are shown. In general, a hazard ratio is a measure of an effect of a co-variate on an outcome of interest over time and a forest plot allows evaluation of the risk probabilities for covariates used in the model. Hazard ratio values less than one indicate a reduction in risk for the particular event. The plotting of a single point for each co-variate risk factor is an easier to interpret visualization as compared the distributed spread of the Shapley plots 30, 32 (FIG. 2). For example, in the ischemic event hazard plot 40, RVD, DAPT duration, and Baseline Hemoglobin all contributed to a lowering of ischemic event risk as their respective hazard ratios were below one (0.721, 0.61, and 0.673, respectively). By contrast, baseline serum creatine and white blood cell count (e.g., measured at baseline) both contributed to increased ischemic event risk as evidenced by the hazard ratios of 1.367 and 1.408, respectively.

In the bleeding event hazard plot 42, the most risk lowering contributing variables were DAPT duration, Baseline Hemoglobin, and RVD with hazard ratios of 0.121, 0.307, and 0.769 respectively. Increased bleeding risk factors included age, baseline serum creatine, baseline white blood cells, and percent diameter stenosis with hazard ratios of 1.493, 1.389, 1.298 and 1.236 respectively.

### Hazard Ratios

The hazard function for an individual patient with the vector of explanatory variables above, xᵢ=(% Diameter stenosis, RVD, DAPT duration, . . . ), can be expressed as:

hᵢ(t)=exp(ƒ(xᵢ))×h₀(t), Equation 1
where:
ƒ is approximated by the machine learning model, in this case by an ensemble of trees, h₀(t) is a baseline hazard value at time t, and i is the i^{th} patient. The function ƒ is decomposed into the equation below through SHAP analysis:

$$f ( {x}_{i} ) = {∅}_{0} + {\sum }_{j = 1}^{p} {∅}_{j} ( f , {x}_{i} ) , \qquad Equation 2$$

where:
Øⱼ(ƒ, xᵢ) is the Shapley value for each explanatory variable for each patient, j is the j^{th} variable, and Ø₀ is a baseline Shapley value. Hence the hazard function can be expressed through Equation 2 as:

hᵢ(t)=exp(Ø₁(ƒ, xᵢ))×exp(Ø₂(ƒ, xᵢ))×exp(Ø₀)×h₀(t).

By averaging exp(Øⱼ(ƒ,xᵢ)) over each patient within two predefined disjoint subgroups (e.g., as 1 vs. 0 for binary variables and greater than or equal to median values vs. below median values for continuous variables) the hazard ratio associated with the variables above can be computed.

The machine learning hazard ratio (HR) is then derived by taking the exponential of Shapley values for the disjoint subgroups as below:

HRⱼ^{ML}=mean_{i∈S}_{₁}(exp(Øⱼ(ƒ, xᵢ))/mean_{i∈S}_{₂}(exp(Øⱼ(ƒ, xᵢ)),
Where:
HRⱼ^{ML}=machine learning derived HR for the explanatory variables above and
S₁=first subgroup of interest
S₂=second (reference) subgroup

### Confidence Intervals

The 95% confidence interval on each of the machine learning derived HR may be calculated using, for example, 1000 bootstraps with replacement and the 2.5^{th} and 97.5^{th} percentile values of the HR values being chosen.

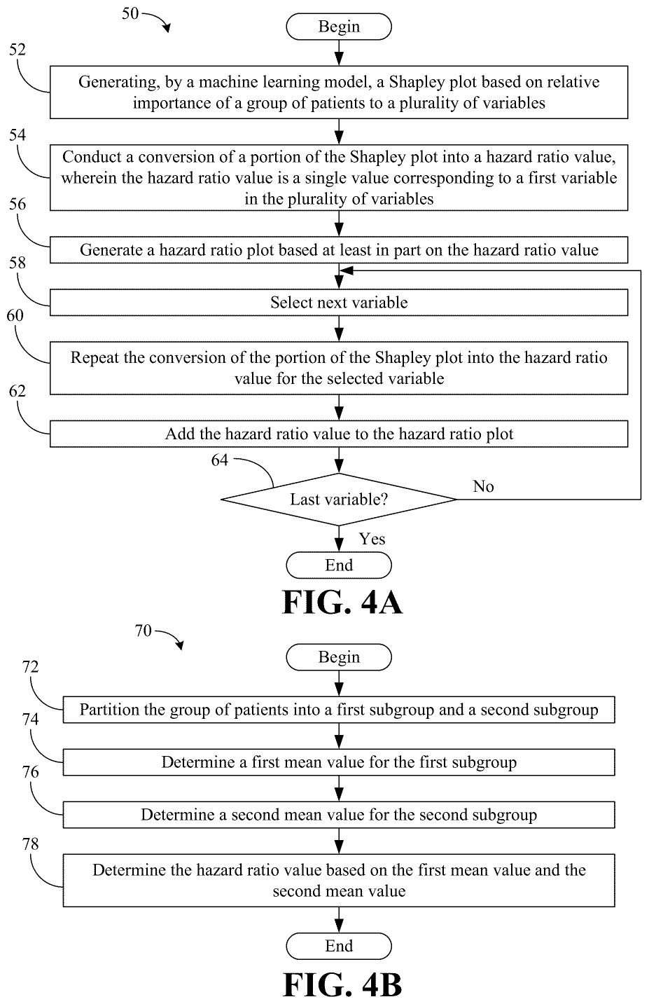

FIG. 4A shows a method 50 of plotting hazard ratios. The method 50 may be implemented in one or more modules as a set of logic instructions stored in a machine- or computer-readable storage medium such as random access memory (RAM), read only memory (ROM), programmable ROM (PROM), firmware, flash memory, etc., in hardware, or any combination thereof. For example, hardware implementations may include configurable logic, fixed-functionality logic, or any combination thereof. Examples of configurable logic (e.g., configurable hardware) include suitably configured programmable logic arrays (PLAs), field programmable gate arrays (FPGAs), complex programmable logic devices (CPLDs), and general purpose microprocessors. Examples of fixed-functionality logic (e.g., fixed-functionality hardware) include suitably configured application specific integrated circuits (ASICs), combinational logic circuits, and sequential logic circuits. The configurable or fixed-functionality logic can be implemented with complementary metal oxide semiconductor (CMOS) logic circuits, transistor-transistor logic (TTL) logic circuits, or other circuits.

Illustrated processing block 52 provides for generating, by a machine learning model, a Shapley plot based on relative importance (e.g., entropy) of a group of patients to a plurality of variables (e.g., percent diameter stenosis, RVD, DAPT duration, etc.). In one embodiment, the relative importance is determined based on a Boruta procedure. The plurality of variables may include one or more binary variables (e.g., male/female) and/or one or more continuous variables (e.g., RVD, DAPT duration). Block 54 conducts a conversion of a portion of the Shapley plot (e.g., percent diameter stenosis portion) into a hazard ratio value, wherein the hazard ratio value is a single value corresponding to a first variable in the plurality of variables. Additionally, block 56 generates a hazard ratio plot based at least in part on the hazard ratio value.

In an embodiment, block 58 selects the next variable (e.g., RVD), wherein block 60 repeats the conversion of the portion of the Shapley plot into the hazard ratio value for the selected variable. Additionally, block 62 adds the hazard ratio value to the hazard ratio plot. A determination may be made at block 64 as to whether the last variable has been reached. If not, the method 50 returns to block 58 and selects the next variable. Thus, the method 50 repeats the conversion of portions of the Shapley plot into hazard ratio values for remaining variables in the plurality of variables to obtain a plurality of hazard ratio values, which are added to the hazard ratio plot. The result is a plot such as, for example, the ischemic event hazard plot 40 (FIG. 3) and/or the bleeding event hazard plot 42 (FIG. 3), already discussed. If it is determined at block 64 that the last variable has been reached, the illustrated method 50 terminates. The method 50 therefore enhances performance at least to the extent that the resulting hazard ratio plot is easier to interpret than the Shapley plot and/or better clinical outcomes are achieved.

FIG. 4B shows a method 70 of converting a Shapley plot into a hazard ratio plot. The method 70 may generally be incorporated into block 54 (FIG. 4A) and/or block 60 (FIG. 4A), already discussed. More particularly, the method 70 may be implemented in one or more modules as a set of logic instructions stored in a machine- or computer-readable storage medium such as RAM, ROM, PROM, firmware, flash memory, etc., in hardware, or any combination thereof.

Illustrated processing block 72 partitions the group of patients into a first subgroup (S₁, e.g., men, patients with a DAPT duration greater than or equal to the median value, etc.) and a second subgroup (S₂, e.g., women, patients with a DAPT duration less than the median value, etc.). Block 74 provides for determining a first mean value for the first subgroup and block 76 determines a second mean value for the second subgroup. In one example, the first mean value and the second mean value are exponential hazard function values (e.g., mean_{i∈S}_{₁} (exp(Øⱼ(ƒ, xᵢ)) and mean_{i∈S}_{₂} (exp(Øⱼ(ƒ, xᵢ)), respectively). In an embodiment, the first mean value and the second mean value are determined based at least in part on a baseline Shapley value (e.g., Ø₀ in Equation 2) and a baseline hazard value (e.g., h₀(t) in Equation 1). Block 78 may determine the hazard ratio value based on the first mean value and the second mean value. In an embodiment, block 78 divides the first mean value by the second mean value.

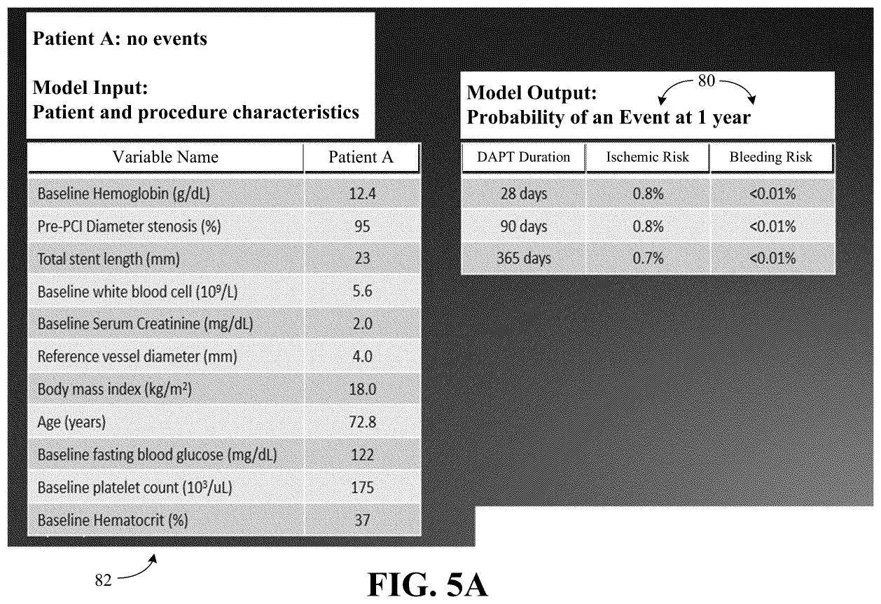

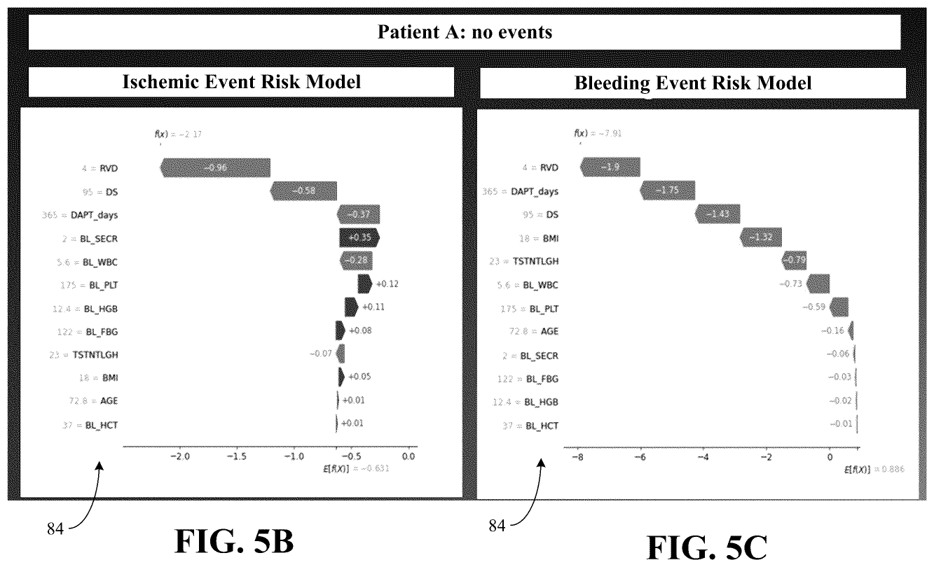

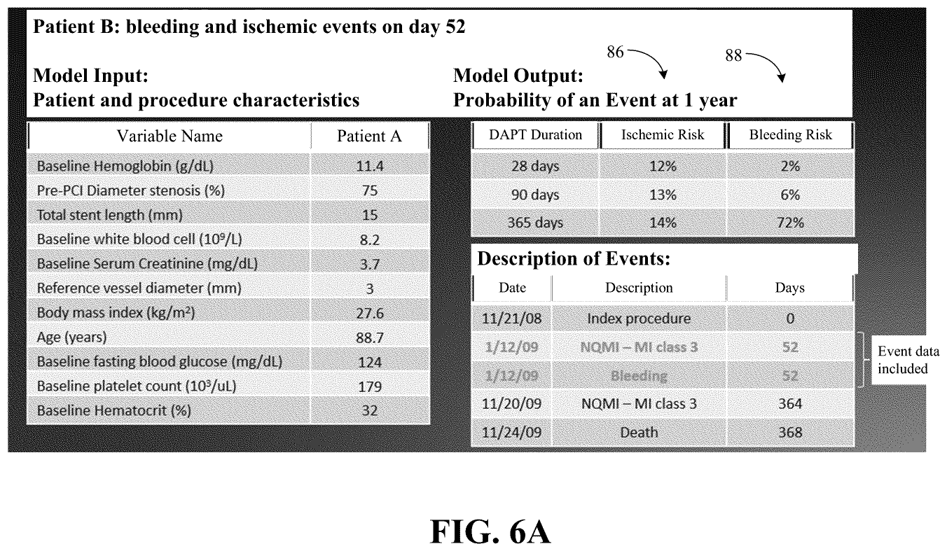

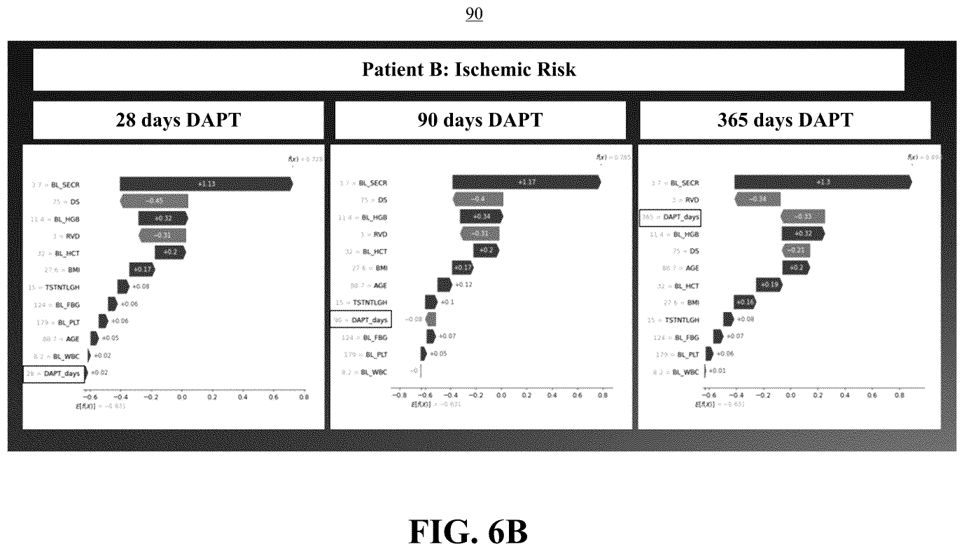

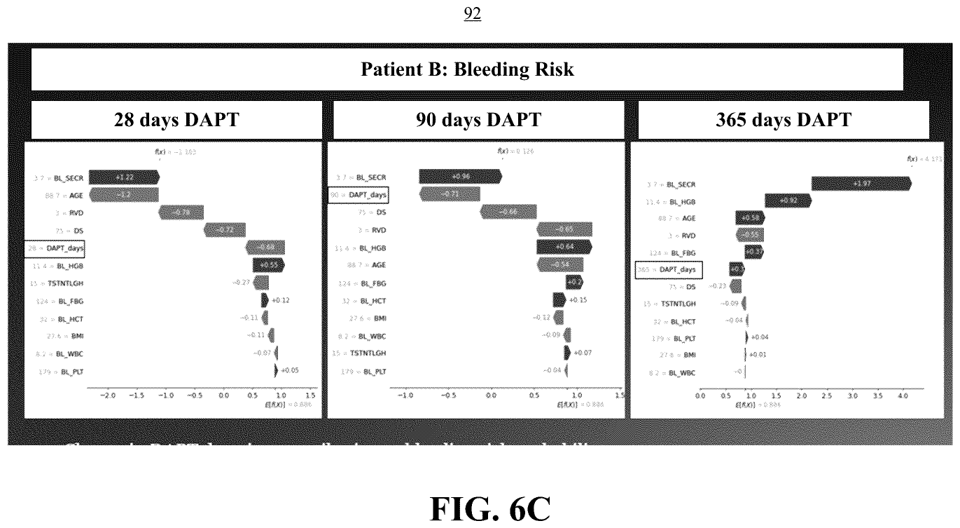

FIGS. 5A-6C demonstrate that once the predictive risk models are built and validated, new patient specific variables can be entered into the models and patient specific risks (e.g., ischemic event and bleeding event risk) can be predicted. As best shown in FIG. 5A, an example of a first patient (“Patient A”) having no health failure events includes the top ten identified variables 82 (e.g., preoperative baseline characteristics). These variables 82 then serve as input for the machine learning model (e.g., Random Survival Forests, Gradient Boosting) and the corresponding risk probabilities 80 (e.g., predictions) for both ischemic and bleeding risk are paired with DAPT durations of 28 days, 90 days and 365 days. In this patient, the ischemic risk was 0.8%, 0.8% and 0.7% for 28 days, 90 days and 365 days of DAPT duration, respectively. Additionally, the bleeding risk was less than 0.01% for all of the same time periods.

As best shown in FIG. 5B, the machine learning model also identifies the patient specific variable components 84 that contribute to the predicted risk. RVD, Diameter stenosis (DS) and DAPT (blue bars) were all factors in lowering future ischemic risk while blood serum creatine (BL_SECR) contributed the largest increase in risk (red bar) with other variables being very minor factors for Patient A. As best shown in FIG. 5C, all ten identified variables contributed to decreased bleeding event risk for Patient A.

As best shown in FIG. 6A, the same ten patient specific variables 82 may be input into the machine learning model for a second patient (“Patient B”) having a bleeding and ischemic even on the 52^{nd} day of receiving DAPT. For this patient, the ischemic risk probabilities 86 are 12%, 13%, and 14% for 28 days, 90 days and 365 days of DAPT duration, respectively. The bleeding risk probabilities 88, however, show a marked increase from 2%, 6%, and 72% for 28 days, 90 days and 365 days of DAPT. This risk prediction indicates a potential for a bleeding event after 90 days that would rise from 6% to 72% if the patient continued DAPT for the full 365 days.

As best shown in a set of charts 90 in FIG. 6B, these increased risk events can be shown from the contributions by the ten patient specific variables at 28 days, 90 days and 365 days of DAPT duration. For ischemic event risk, blood serum creatine (BL_SECR) was the largest risk factor for all three time periods but the value of blood serum creatine does not change significantly from 1.13, 1.17, and 1.3 over the 28 days, 90 days and 365 days of DAPT duration, respectively. All the other nine factors were relatively small contributors, and their values did not appreciably change over the measured period.

As best shown in a set of charts 92 in FIG. 6C, bleeding risk for Patient B showed changes in the contributing variables. Blood serum creatine (BL_SECR) was the largest risk factor for all three time periods and the value of blood serum creatine rose to 1.97 at 365 days DAPT duration from 1.22, 0.96, during the earlier 28 days and 90 days DAPT durations, respectively. Additionally, blood hemoglobin (BL_HBG) and age rose to become increased bleeding factors at 365 days DAPT duration while concomitantly many other variables (e.g., DAPT days, DS, RVD and Age) showed less bleeding risk contributions. Additionally, DAPT days transitioned from negative to positive when the duration was extended from 90 days to 365 days.

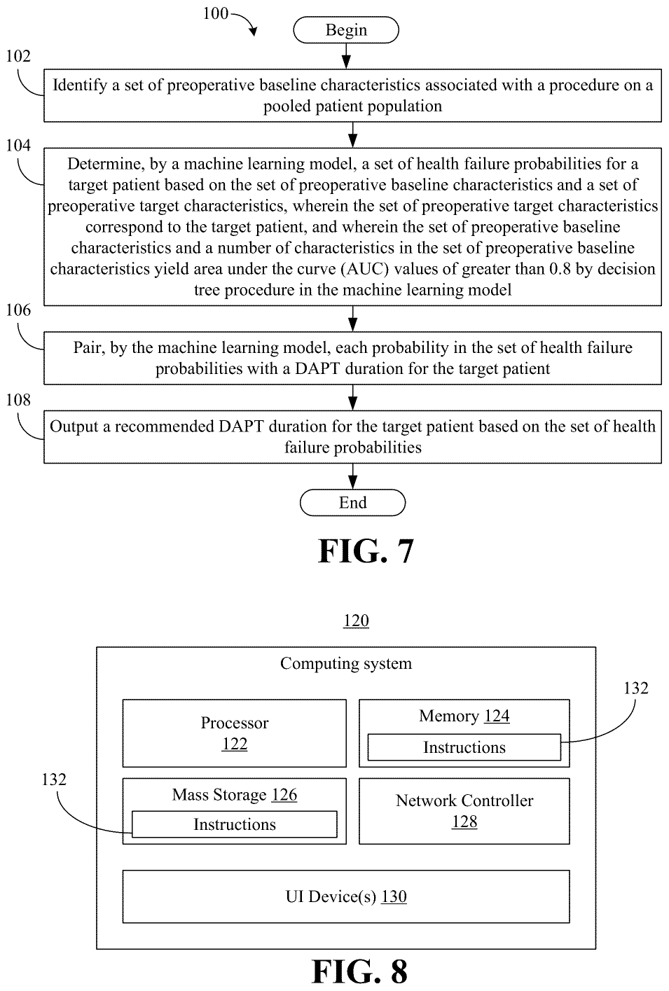

FIG. 7 shows a method 100 of recommending DAPT durations. The method 100 may generally be implemented in conjunction with the method 50 (FIG. 4A) and/or the method 70 (FIG. 4B), already discussed. More particularly, the method may be implemented in one or more modules as a set of logic instructions stored in a machine- or computer-readable storage medium such as RAM, ROM, PROM, firmware, flash memory, etc., in hardware, or any combination thereof.

Illustrated processing block 102 provides for identifying a set of preoperative baseline characteristics associated with a procedure on a pooled patient population. In one example, the procedure is a stent procedure. Block 104 determines, by a machine learning model, a set of health failure probabilities for a target patient based on the set of preoperative baseline characteristics and a set of preoperative target characteristics, wherein the set of preoperative target characteristics correspond to the target patient. In an embodiment, the health failure probabilities are associated with a time to first ischemic event and/or a time to first bleeding event. Moreover, the machine learning model may include a Random Survival Forests model, a Gradient Boosting model, etc., or any combination thereof.

For example, Gradient Boosting works on an ensemble technique called “boosting”. Like other boosting models, a Gradient boost sequentially combines many weak learners to form a strong learner. Typically, Gradient Boosting uses decision trees as weak learners. The idea of boosting is to train weak learners sequentially, each trying to correct its respective predecessor. Accordingly, boosting will always learn something that is not completely accurate but a small step in the correct direction at each learning phase. As the procedure moves forward by sequentially correcting the previous errors, the prediction power is improved. Sequentially combining weak trees to form a strong tree, improves the accuracy of the model (e.g., achieving low bias and low variance).

Block 106 pairs, by the machine learning model, each probability in the set of health failure probabilities with a postoperative DAPT duration (e.g., 28 days, 90 days, 365 days) for the target patient. Additionally, block 108 may output a recommended DAPT duration for the target patient based on the set of health failure probabilities. For example, the recommended DAPT duration might correspond to the lowest probability in the set of health failure probabilities. The method 100 therefore enhances performance at least to the extent that the time based prediction produces better health outcomes in the context of stent procedures.

Turning now to FIG. 8, a performance-enhanced computing system 120 is shown. In the illustrated example, the computing system 120 includes a processor 122, a memory 124 (e.g., volatile memory such as, for example, DRAM), mass storage 126 (e.g., persistent or non-volatile memory such as, for example, ROM, flash memory, solid state drive/SSD, hard disk drive/HDD), a network controller 128 (e.g., wired and/or wireless) and one or more user interface devices 130 (e.g., display, speaker). In an embodiment, the memory 124 and/or the mass storage 126 include a set of instructions 132, which when executed by the processor 122, cause the processor 122 and/or the computing system 120 to perform one or more aspects of the method 50 (FIG. 4A), the method 70 (FIG. 4B) and/or the method 100 (FIG. 7), already discussed.

Thus, execution of the instructions 132 may cause the processor 122 and/or the computing system 120 to generate, by a machine learning model, a Shapley plot based on average marginal contributions of a group of patients to a plurality of variables, conduct a conversion of a portion of the Shapley plot into a hazard ratio value, wherein the hazard ratio value is a single value corresponding to a first variable in the plurality of variables, and generate a hazard ratio plot based at least in part on the hazard ratio value. The computing system 120 is therefore considered performance-enhanced at least to the extent that the resulting hazard ratio plot is easier to interpret than the Shapley plot and/or better clinical outcomes are achieved.

Execution of the instructions 132 may also cause the processor 122 and/or the computing system to identify a set of preoperative baseline characteristics associated with a procedure on a pooled patient population, determine, by a machine learning model, a set of health failure probabilities for a target patient based on the set of preoperative baseline characteristic and a set of preoperative target characteristics, wherein the set of preoperative target characteristics correspond to the target client, and pair, by the machine learning model, each probability in the set of health failure probabilities with a postoperative dual antiplatelet therapy (DAPT) duration for the target patient. The computing system 120 is therefore further considered performance-enhanced at least to the extent that the time based prediction produces better health outcomes in the context of stent procedures.

### Results

Once the top ten variables were identified, the analysis of survival data for identifying differences in survival analysis was compared between the technology described herein and a traditional Cox proportional hazards model. The Cox statistical regression model is widely used in clinical trials to investigate the simultaneous effects of several predictor variables (co-variates) and the time a specified event takes to happen.

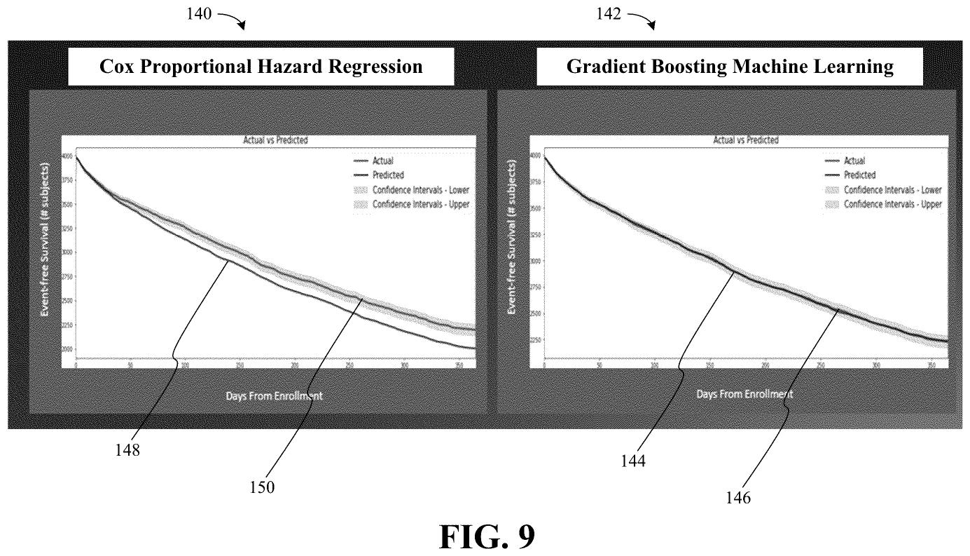

FIG. 9 shows a conventional chart 140 when the top ten covariates for ischemic events were used as input data for the Cox Proportional hazard regression and an enhanced chart 142 when the top ten covariates for ischemic events were used as input data for the Gradient boosting machine learning models described herein. It can be observed that the Gradient Boosting machine learning model for ischemic events was very accurate as a predicted events curve 144 corresponded almost identically being overlayed with an actual event rates curve 146. By contrast, a predicted event rate curve 148 for the Cox Proportional Hazard regression is so under estimated as compared an actual curve 150 that the curve 148 falls outside the confidence interval.

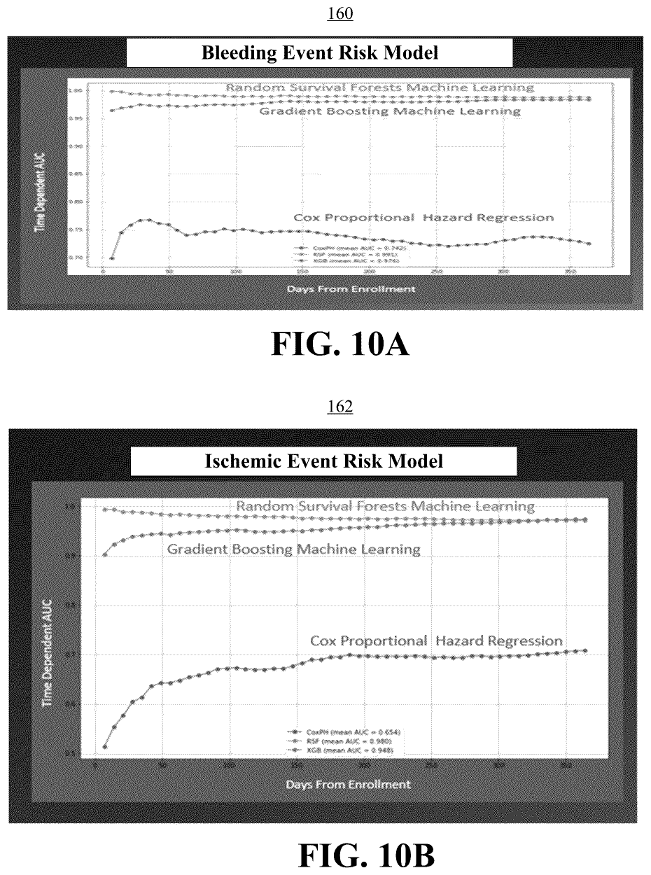

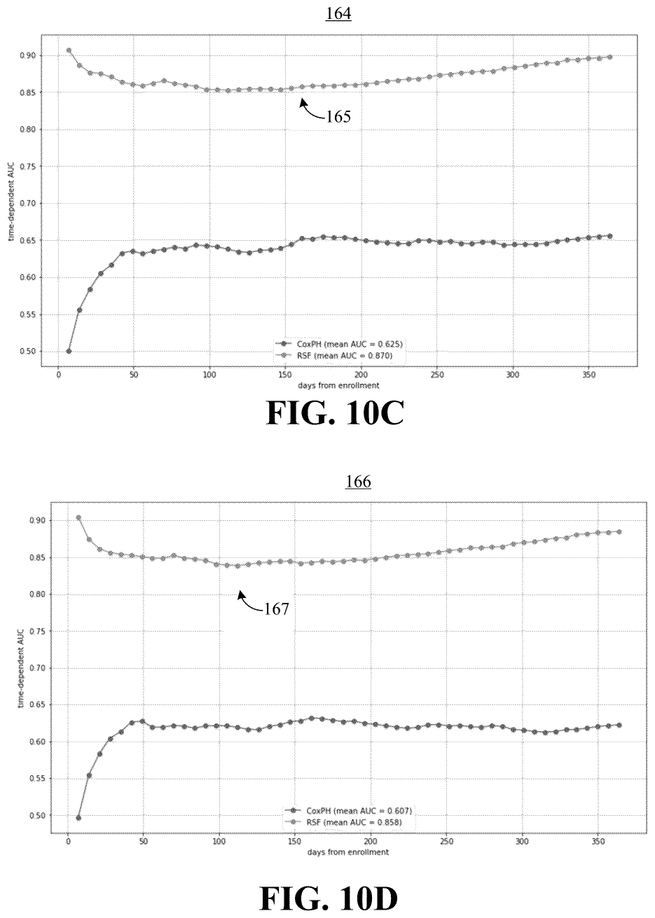

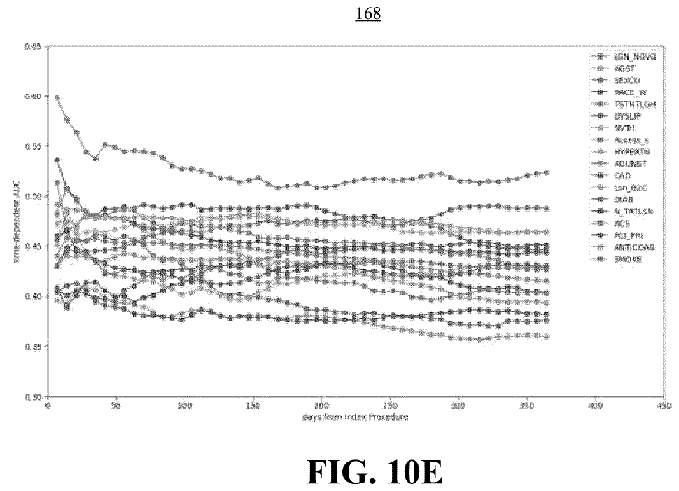

FIGS. 10A and 10B further demonstrate how well the technology described herein performs by calculating the time-dependent Area Under the Curve (tAUC), which is a measure of the predictive classification and performance. In the illustrated example, the top eight preoperative baseline characteristics were used to determine the health failure probabilities. The Random Survival Forest and Gradient Boosting machine learning models demonstrated significantly superior tAUCs (e.g., values of greater than 0.9 by decision tree procedure) as compared to Cox Proportional Hazard regression in both an ischemic event chart 162 and a bleeding event chart 160. A higher tAUC value indicates enhanced performance.

FIG. 10C shows a chart 164 in which the top four preoperative baseline characteristics were used to determine the health failure probabilities. In the illustrated example, the set of preoperative baseline characteristics and the number of characteristics in the set of preoperative baseline characteristics yield AUC values 165 of greater than 0.85 by decision tree procedure in the machine learning model.

FIG. 10D shows a chart 166 in which the top three preoperative baseline characteristics were used to determine the health failure probabilities. In the illustrated example, the set of preoperative baseline characteristics and the number of characteristics in the set of preoperative baseline characteristics yield AUC values 167 of greater than 0.8 by decision tree procedure in the machine learning model.

FIG. 10E shows a chart 168 of individual risk scores for a plurality of variables. The chart 168 demonstrates the discriminative ability of individual variables and the estimated time-dependent AUC over time.

### ADDITIONAL NOTES AND EXAMPLES

Example 1 includes a performance-enhanced computing system comprising a processor, and a memory coupled to the processor, the memory including a set of instructions, which when executed by the processor, cause the computing system to identify a set of preoperative baseline characteristics associated with a procedure on a pooled patient population, determine, by a machine learning model, a set of health failure probabilities for a target patient based on the set of preoperative baseline characteristics and a set of preoperative target characteristics, wherein the set of preoperative target characteristics correspond to the target patient, and wherein the set of preoperative baseline characteristics and a number of characteristics in the set of preoperative baseline characteristics yield area under the curve (AUC) values of greater than 0.8 by decision tree procedure in the machine learning model, and pair, by the machine learning model, each probability in the set of health failure probabilities with a postoperative dual antiplatelet therapy (DAPT) duration for the target patient.

Example 2 includes the computing system of Example 1, wherein the instructions, when executed, further cause the computing system to output a recommended DAPT duration for the target patient based on the set of health failure probabilities.

Example 3 includes the computing system of Example 2, wherein the recommended DAPT duration is to correspond to a lowest probability in the set of health failure probabilities.

Example 4 includes the computing system of Example 1, wherein the set of health failure probabilities are to be associated with a time to first ischemic event.

Example 5 includes the computing system of Example 1, wherein the set of health failure probabilities are to be associated with a time to first bleeding event.

Example 6 includes the computing system of Example 1, wherein the machine learning model is to be a Random Survival Forests model.

Example 7 includes the computing system of Example 1, wherein the machine learning model is to be a Gradient Boosting model.

Example 8 includes the computing system of Example 1, wherein the procedure is to be a stent procedure.

Example 9 includes the computing system of any one of Examples 1 to 8, wherein the set of preoperative baseline characteristics and the number of characteristics in the set of preoperative baseline characteristics yield AUC values of greater than 0.85 by decision tree procedure in the machine learning model.

Example 10 includes the computing system of Example 9, wherein the set of preoperative baseline characteristics and the number of characteristics in the set of preoperative baseline characteristics yield AUC values of greater than 0.9 by decision tree procedure in the machine learning model.

Example 11 includes at least one computer readable storage medium comprising a set of instructions, which when executed by a computing system, cause the computing system to identify a set of preoperative baseline characteristics associated with a procedure on a pooled patient population, determine, by a machine learning model, a set of health failure probabilities for a target patient based on the set of preoperative baseline characteristics and a set of preoperative target characteristics, wherein the set of preoperative target characteristics correspond to the target patient, and wherein the set of preoperative baseline characteristics and a number of characteristics in the set of preoperative baseline characteristics yield area under the curve (AUC) values of greater than 0.8 by decision tree procedure in the machine learning model, and pair, by the machine learning model, each probability in the set of health failure probabilities with a postoperative dual antiplatelet therapy (DAPT) duration for the target patient.

Example 12 includes the at least one computer readable storage medium of Example 11, wherein the instructions, when executed, further cause the computing system to output a recommended DAPT duration for the target patient based on the set of health failure probabilities.

Example 13 includes the at least one computer readable storage medium of Example 12, wherein the recommended DAPT duration is to correspond to a lowest probability in the set of health failure probabilities.

Example 14 includes the at least one computer readable storage medium of Example 11, wherein the set of health failure probabilities are to be associated with a time to first ischemic event.

Example 15 includes the at least one computer readable storage medium of Example 11, wherein the set of health failure probabilities are to be associated with a time to first bleeding event.

Example 16 includes the at least one computer readable storage medium of Example 11, wherein the machine learning model is to be a Random Survival Forests model.

Example 17 includes the at least one computer readable storage medium of Example 11, wherein the machine learning model is to be a Gradient Boosting model.

Example 18 includes the at least one computer readable storage medium of Example 11, wherein the procedure is to be a stent procedure.

Example 19 includes the at least one computer readable storage medium of any one of Examples 11 to 18, wherein the set of preoperative baseline characteristics and the number of characteristics in the set of preoperative baseline characteristics yield AUC values of greater than 0.85 by decision tree procedure in the machine learning model.

Example 20 includes the at least one computer readable storage medium of Example 19, wherein the set of preoperative baseline characteristics and the number of characteristics in the set of preoperative baseline characteristics yield AUC values of greater than 0.9 by decision tree procedure in the machine learning model.

Example 21 includes a method of operating a performance-enhanced computing system, the method comprising identifying a set of preoperative baseline characteristics associated with a procedure on a pooled patient population, determining, by a machine learning model, a set of health failure probabilities for a target patient based on the set of preoperative baseline characteristics and a set of preoperative target characteristics, wherein the set of preoperative target characteristics correspond to the target patient, and wherein the set of preoperative baseline characteristics and a number of characteristics in the set of preoperative baseline characteristics yield area under the curve (AUC) values of greater than 0.8 by decision tree procedure in the machine learning model, and pairing, by the machine learning model, each probability in the set of health failure probabilities with a postoperative dual antiplatelet therapy (DAPT) duration for the target patient.

Example 22 includes the method of Example 21, further including outputting a recommended DAPT duration for the target patient based on the set of health failure probabilities.

Example 23 includes the method of Example 22, wherein the recommended DAPT duration corresponds to a lowest probability in the set of health failure probabilities.

Example 24 includes the method of Example 21, wherein the set of health failure probabilities are associated with a time to first ischemic event.

Example 25 includes the method of Example 21, wherein the set of health failure probabilities are associated with a time to first bleeding event.

Example 26 includes the method of Example 21, wherein the machine learning model is a Random Survival Forests model.

Example 27 includes the method of Example 21, wherein the machine learning model is a Gradient Boosting model.

Example 28 includes the method of Example 21, wherein the procedure is a stent procedure.

Example 29 includes the method of any one of Examples 21 to 28, wherein the set of preoperative baseline characteristics and the number of characteristics in the set of preoperative baseline characteristics yield AUC values of greater than 0.85 by decision tree procedure in the machine learning model.

Example 30 includes the method of Example 29, wherein the set of preoperative baseline characteristics and the number of characteristics in the set of preoperative baseline characteristics yield AUC values of greater than 0.9 by decision tree procedure in the machine learning model.

Example 31 includes a performance-enhanced computing system comprising a processor, and a memory coupled to the processor, the memory including a set of instructions, which when executed by the processor, cause the computing system to generate, by a machine learning model, a Shapley plot based on relative importance of a group of patients to a plurality of variables, conduct a conversion of a portion of the Shapley plot into a hazard ratio value, wherein the hazard ratio value is a single value corresponding to a first variable in the plurality of variables, and generate a hazard ratio plot based at least in part on the hazard ratio value.

Example 32 includes the computing system of Example 31, wherein the instructions, when executed, further cause the computing system to repeat the conversion of the portion of the Shapley plot into the hazard ratio value for remaining variables in the plurality of variables to obtain a plurality of hazard ratio values, and add the plurality of hazard ratio values to the hazard ratio plot.

Example 33 includes the computing system of Example 31, wherein to conduct the conversion of the portion of the Shapley plot into the hazard value, the instructions, when executed, further cause the computing system to partition the group of patients into a first subgroup and a second subgroup, determine a first mean value for the first subgroup, determine a second mean value for the second subgroup, and determine the hazard ratio value based on the first mean value and the second mean value.

Example 34 includes the computing system of Example 33, wherein the first mean value and the second mean value are to be exponential hazard function values.

Example 35 includes the computing system of Example 33, wherein the first mean value and the second mean value are determined based at least in part on a baseline Shapley value and a baseline hazard value.

Example 36 includes the computing system of any one of Examples 31 to 35, wherein the plurality of variables are to include one or more binary variables.

Example 37 includes the computing system of any one of Examples 31 to 35, wherein the plurality of variables are to include one or more continuous variables.

Example 38 includes at least one computer readable storage medium comprising a set of instructions, which when executed by a computing system, cause the computing system to generate, by a machine learning model, a Shapley plot based on relative importance of a group of patients to a plurality of variables, conduct a conversion of a portion of the Shapley plot into a hazard ratio value, wherein the hazard ratio value is a single value corresponding to a first variable in the plurality of variables, and generate a hazard ratio plot based at least in part on the hazard ratio value.

Example 39 includes the at least one computer readable storage medium of Example 38, wherein the instructions, when executed, further cause the computing system to repeat the conversion of the portion of the Shapley plot into the hazard ratio value for remaining variables in the plurality of variables to obtain a plurality of hazard ratio values, and add the plurality of hazard ratio values to the hazard ratio plot.

Example 40 includes the at least one computer readable storage medium of Example 38, wherein to conduct the conversion of the portion of the Shapley plot into the hazard value, the instructions, when executed, further cause the computing system to partition the group of patients into a first subgroup and a second subgroup, determine a first mean value for the first subgroup, determine a second mean value for the second subgroup, and determine the hazard ratio value based on the first mean value and the second mean value.

Example 41 includes the at least one computer readable storage medium of Example 40, wherein the first mean value and the second mean value are to be exponential hazard function values.

Example 42 includes the at least one computer readable storage medium of Example 40, wherein the first mean value and the second mean value are determined based at least in part on a baseline Shapley value and a baseline hazard value.

Example 43 includes the at least one computer readable storage medium of any one of Examples 38 to 42, wherein the plurality of variables are to include one or more binary variables.

Example 44 includes the at least one computer readable storage medium of any one of Examples 38 to 42, wherein the plurality of variables are to include one or more continuous variables.

Example 45 includes a method of operating a performance-enhanced computing system, the method comprising generating, by a machine learning model, a Shapley plot based on relative importance of a group of patients to a plurality of variables, conducting a conversion of a portion of the Shapley plot into a hazard ratio value, wherein the hazard ratio value is a single value corresponding to a first variable in the plurality of variables, and generating a hazard ratio plot based at least in part on the hazard ratio value.

Example 46 includes the method of Example 45, further including repeating the conversion of the portion of the Shapley plot into the hazard ratio value for remaining variables in the plurality of variables to obtain a plurality of hazard ratio values, and adding the plurality of hazard ratio values to the hazard ratio plot.

Example 47 includes the method of Example 45, wherein conducting the conversion of the portion of the Shapley plot into the hazard ratio value includes partitioning the group of patients into a first subgroup and a second subgroup, determining a first mean value for the first subgroup, determining a second mean value for the second subgroup, and determining the hazard ratio value based on the first mean value and the second mean value.

Example 48 includes the method of Example 47, wherein the first mean value and the second mean value are exponential hazard function values.

Example 49 includes the method of Example 47, wherein the first mean value and the second mean value are determined based at least in part on a baseline Shapley value and a baseline hazard value.

Example 50 includes the method of any one of Examples 45 to 49, wherein the plurality of variables include one or more binary variables.

Example 51 includes the method of any one of Examples 45 to 49, wherein the plurality of variables include one or more continuous variables.

Example 52 includes an apparatus comprising means for performing the method of any one of Examples 21 to 30.

Example 53 includes an apparatus comprising means for performing the method of any one of Examples 45 to 51.

Embodiments are applicable for use with all types of semiconductor integrated circuit (“IC”) chips. Examples of these IC chips include but are not limited to processors, controllers, chipset components, programmable logic arrays (PLAs), memory chips, network chips, systems on chip (SoCs), SSD (solid state drive)/NAND controller ASICs, and the like. In addition, in some of the drawings, signal conductor lines are represented with lines. Some may be different, to indicate more constituent signal paths, have a number label, to indicate a number of constituent signal paths, and/or have arrows at one or more ends, to indicate primary information flow direction. This, however, should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments are not limited to the same. As manufacturing techniques (e.g., photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments. Further, arrangements may be shown in block diagram form in order to avoid obscuring embodiments, and also in view of the fact that specifics with respect to implementation of such block diagram arrangements are highly dependent upon the platform within which the embodiment is to be implemented, i.e., such specifics should be well within purview of one skilled in the art. Where specific details (e.g., circuits) are set forth in order to describe example embodiments, it should be apparent to one skilled in the art that embodiments can be practiced without, or with variation of, these specific details. The description is thus to be regarded as illustrative instead of limiting.

The term “coupled” may be used herein to refer to any type of relationship, direct or indirect, between the components in question, and may apply to electrical, mechanical, fluid, optical, electromagnetic, electromechanical or other connections. In addition, the terms “first”, “second”, etc. may be used herein only to facilitate discussion, and carry no particular temporal or chronological significance unless otherwise indicated.

As used in this application and in the claims, a list of items joined by the term “one or more of” may mean any combination of the listed terms. For example, the phrases “one or more of A, B or C” may mean A; B; C; A and B; A and C; B and C; or A, B and C.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

We claim:

1. A computing system comprising:
a processor; and
a memory coupled to the processor, the memory including a set of instructions, which when executed by the processor, cause the computing system to:
identify a set of preoperative baseline characteristics associated with a procedure on a pooled patient population;
determine, by a machine learning model, a set of health failure probabilities for a target patient based on the set of preoperative baseline characteristics and a set of preoperative target characteristics, wherein the set of preoperative target characteristics correspond to the target patient, and wherein the set of preoperative baseline characteristics and a number of characteristics in the set of preoperative baseline characteristics yield area under the curve (AUC) values of greater than 0.8 by decision tree procedure in the machine learning model; and
pair, by the machine learning model, each probability in the set of health failure probabilities with a postoperative dual antiplatelet therapy (DAPT) duration for the target patient.

2. The computing system of claim 1, wherein the instructions, when executed, further cause the computing system to output a recommended DAPT duration for the target patient based on the set of health failure probabilities.

3. The computing system of claim 2, wherein the recommended DAPT duration is to correspond to a lowest probability in the set of health failure probabilities.

4. The computing system of claim 1, wherein the set of health failure probabilities are to be associated with a time to first ischemic event.

5. The computing system of claim 1, wherein the set of health failure probabilities are to be associated with a time to first bleeding event.

6. The computing system of claim 1, wherein the machine learning model is to be a Random Survival Forests model.

7. The computing system of claim 1, wherein the machine learning model is to be a Gradient Boosting model.

8. The computing system of claim 1, wherein the procedure is to be a stent procedure.

9. The computing system of claim 1, wherein the set of preoperative baseline characteristics and the number of characteristics in the set of preoperative baseline characteristics yield AUC values of greater than 0.85 by decision tree procedure in the machine learning model.

10. The computing system of claim 9, wherein the set of preoperative baseline characteristics and the number of characteristics in the set of preoperative baseline characteristics yield AUC values of greater than 0.9 by decision tree procedure in the machine learning model.

11. At least one computer readable storage medium comprising a set of instructions, which when executed by a computing system, cause the computing system to:
identify a set of preoperative baseline characteristics associated with a procedure on a pooled patient population;
determine, by a machine learning model, a set of health failure probabilities for a target patient based on the set of preoperative baseline characteristics and a set of preoperative target characteristics, wherein the set of preoperative target characteristics correspond to the target patient, and wherein the set of preoperative baseline characteristics and a number of characteristics in the set of preoperative baseline characteristics yield area under the curve (AUC) values of greater than 0.8 by decision tree procedure in the machine learning model; and
pair, by the machine learning model, each probability in the set of health failure probabilities with a postoperative dual antiplatelet therapy (DAPT) duration for the target patient.

12. The at least one computer readable storage medium of claim 11, wherein the instructions, when executed, further cause the computing system to output a recommended DAPT duration for the target patient based on the set of health failure probabilities.

13. The at least one computer readable storage medium of claim 12, wherein the recommended DAPT duration is to correspond to a lowest probability in the set of health failure probabilities.

14. The at least one computer readable storage medium of claim 11, wherein the set of health failure probabilities are to be associated with a time to first ischemic event.

15. The at least one computer readable storage medium of claim 11, wherein the set of health failure probabilities are to be associated with a time to first bleeding event.

16. The at least one computer readable storage medium of claim 11, wherein the machine learning model is to be a Random Survival Forests model.

17. The at least one computer readable storage medium of claim 11, wherein the machine learning model is to be a Gradient Boosting model.

18. The at least one computer readable storage medium of claim 11, wherein the procedure is to be a stent procedure.

19. The at least one computer readable storage medium of claim 11, wherein the set of preoperative baseline characteristics and the number of characteristics in the set of preoperative baseline characteristics yield AUC values of greater than 0.85 by decision tree procedure in the machine learning model.

20. The at least one computer readable storage medium of claim 19, wherein the set of preoperative baseline characteristics and the number of characteristics in the set of preoperative baseline characteristics yield AUC values of greater than 0.9 by decision tree procedure in the machine learning model.

21. A method comprising:
identifying a set of preoperative baseline characteristics associated with a procedure on a pooled patient population;
determining, by a machine learning model, a set of health failure probabilities for a target patient based on the set of preoperative baseline characteristics and a set of preoperative target characteristics, wherein the set of preoperative target characteristics correspond to the target patient, and wherein the set of preoperative baseline characteristics and a number of characteristics in the set of preoperative baseline characteristics yield area under the curve (AUC) values of greater than 0.8 by decision tree procedure in the machine learning model; and
pairing, by the machine learning model, each probability in the set of health failure probabilities with a postoperative dual antiplatelet therapy (DAPT) duration for the target patient.

22. The method of claim 21, further including outputting a recommended DAPT duration for the target patient based on the set of health failure probabilities.

23. The method of claim 22, wherein the recommended DAPT duration corresponds to a lowest probability in the set of health failure probabilities.

24. The method of claim 21, wherein the set of health failure probabilities are associated with a time to first ischemic event.

25. The method of claim 21, wherein the set of health failure probabilities are associated with a time to first bleeding event.

26. The method of claim 21, wherein the machine learning model is a Random Survival Forests model.

27. The method of claim 21, wherein the machine learning model is a Gradient Boosting model.

28. The method of claim 21, wherein the procedure is a stent procedure.

29. The method of claim 21, wherein the set of preoperative baseline characteristics and the number of characteristics in the set of preoperative baseline characteristics yield AUC values of greater than 0.85 by decision tree procedure in the machine learning model.

30. The method of claim 29, wherein the set of preoperative baseline characteristics and the number of characteristics in the set of preoperative baseline characteristics yield AUC values of greater than 0.9 by decision tree procedure in the machine learning model.

31. A computing system comprising:
a processor; and
a memory coupled to the processor, the memory including a set of instructions, which when executed by the processor, cause the computing system to:
generate, by a machine learning model, a Shapley plot based on relative importance of a group of patients to a plurality of variables;
conduct a conversion of a portion of the Shapley plot into a hazard ratio value, wherein the hazard ratio value is a single value corresponding to a first variable in the plurality of variables; and
generate a hazard ratio plot based at least in part on the hazard ratio value.

32. The computing system of claim 31, wherein the instructions, when executed, further cause the computing system to:
repeat the conversion of the portion of the Shapley plot into the hazard ratio value for remaining variables in the plurality of variables to obtain a plurality of hazard ratio values; and
add the plurality of hazard ratio values to the hazard ratio plot.

33. The computing system of claim 31, wherein to conduct the conversion of the portion of the Shapley plot into the hazard value, the instructions, when executed, further cause the computing system to:
partition the group of patients into a first subgroup and a second subgroup;
determine a first mean value for the first subgroup;
determine a second mean value for the second subgroup; and
determine the hazard ratio value based on the first mean value and the second mean value.

34. The computing system of claim 33, wherein the first mean value and the second mean value are to be exponential hazard function values.

35. The computing system of claim 33, wherein the first mean value and the second mean value are determined based at least in part on a baseline Shapley value and a baseline hazard value.

36. The computing system of claim 31, wherein the plurality of variables are to include one or more binary variables.

37. The computing system of claim 31, wherein the plurality of variables are to include one or more continuous variables.

38. At least one computer readable storage medium comprising a set of instructions, which when executed by a computing system, cause the computing system to:
generate, by a machine learning model, a Shapley plot based on relative importance of a group of patients to a plurality of variables;
conduct a conversion of a portion of the Shapley plot into a hazard ratio value, wherein the hazard ratio value is a single value corresponding to a first variable in the plurality of variables; and
generate a hazard ratio plot based at least in part on the hazard ratio value.

39. The at least one computer readable storage medium of claim 38, wherein the instructions, when executed, further cause the computing system to:
repeat the conversion of the portion of the Shapley plot into the hazard ratio value for remaining variables in the plurality of variables to obtain a plurality of hazard ratio values; and
add the plurality of hazard ratio values to the hazard ratio plot.

40. The at least one computer readable storage medium of claim 38, wherein to conduct the conversion of the portion of the Shapley plot into the hazard value, the instructions, when executed, further cause the computing system to:
partition the group of patients into a first subgroup and a second subgroup;
determine a first mean value for the first subgroup;
determine a second mean value for the second subgroup; and
determine the hazard ratio value based on the first mean value and the second mean value.

41. The at least one computer readable storage medium of claim 40, wherein the first mean value and the second mean value are to be exponential hazard function values.

42. The at least one computer readable storage medium of claim 40, wherein the first mean value and the second mean value are determined based at least in part on a baseline Shapley value and a baseline hazard value.

43. The at least one computer readable storage medium of claim 38, wherein the plurality of variables are to include one or more binary variables.

44. The at least one computer readable storage medium of claim 38, wherein the plurality of variables are to include one or more continuous variables.

45. A method comprising:
generating, by a machine learning model, a Shapley plot based on relative importance of a group of patients to a plurality of variables;
conducting a conversion of a portion of the Shapley plot into a hazard ratio value, wherein the hazard ratio value is a single value corresponding to a first variable in the plurality of variables; and
generating a hazard ratio plot based at least in part on the hazard ratio value.

46. The method of claim 45, further including:
repeating the conversion of the portion of the Shapley plot into the hazard ratio value for remaining variables in the plurality of variables to obtain a plurality of hazard ratio values; and
adding the plurality of hazard ratio values to the hazard ratio plot.

47. The method of claim 45, wherein conducting the conversion of the portion of the Shapley plot into the hazard ratio value includes:
partitioning the group of patients into a first subgroup and a second subgroup;
determining a first mean value for the first subgroup;
determining a second mean value for the second subgroup; and
determining the hazard ratio value based on the first mean value and the second mean value.

48. The method of claim 47, wherein the first mean value and the second mean value are exponential hazard function values.

49. The method of claim 47, wherein the first mean value and the second mean value are determined based at least in part on a baseline Shapley value and a baseline hazard value.

50. The method of claim 45, wherein the plurality of variables include one or more binary variables.

51. The method of claim 45, wherein the plurality of variables include one or more continuous variables.
